(19) United States
(12) Patent Application Publication
(10) Pub. No.: US 2017/0290549 A1
(43) Pub. Date: Oct. 12, 2017

(54) SYSTEMS AND METHODS FOR VARIABLE FILTER ADJUSTMENT BY HEART RATE METRIC FEEDBACK AND NOISE REFERENCE SENSOR

(71) Applicant: Valencell, Inc., Raleigh, NC (US)

(72) Inventor: Eric Douglas ROMESBURG, Chapel Hill, NC (US)

(21) Appl. No.: 15/477,706

(22) Filed: Apr. 3, 2017

Related U.S. Application Data
(60) Provisional application No. 62/321,320, filed on Apr. 12, 2016.

(51) Int. Cl. A61B 5/00; A61B 7/00; A61B 5/0402; A61B 5/0205; A61B 5/0476

(52) U.S. Cl. CPC A61B 5/721; A61B 5/0205; A61B 5/725; A61B 5/0476; A61B 5/0402; A61B 5/0095; A61B 7/00; A61B 2562/0219; A61B 2562/0247; A61B 5/0816

(57) ABSTRACT

A physiological signal processing system/method for a physiological waveform that includes a cardiovascular signal component provides a first variable high pass filter that is responsive to the physiological waveform, and to a first corner frequency that is applied. A second variable high pass filter is responsive to a noise reference waveform from a noise reference sensor and is configured to high pass filter the noise reference waveform in response to a second corner frequency that is applied. A heart rate metric extractor is responsive to the variable high pass filters and is configured to extract a heart rate metric from the physiological waveform that is high pass filtered. A corner frequency adjuster is responsive to the heart rate metric extractor and is configured to determine the corner frequencies that are applied to the variable high pass filters, based on the heart rate metric that was extracted.

### CLAIM OF PRIORITY

This application claims the benefit of provisional Patent Application No. 62/321,320, filed Apr. 12, 2016, entitled Systems and Methods for Variable Filter Adjustment by Heart Rate Metric Feedback and Noise Reference Sensor, assigned to the assignee of the present invention, the disclosure of which is hereby incorporated herein by reference in their entirety as if set forth fully herein.

### BACKGROUND

Various embodiments described herein relate generally to signal processing systems and methods, and more particularly to physiological signal processing systems and methods.

There is a growing market demand for personal health and environmental monitors, for example, for gauging overall health, fitness, metabolism, and vital status during exercise, athletic training, work, public safety activities, dieting, daily life activities, sickness and physical therapy. These personal health and environmental monitors process physiological signals that may be obtained from one or more physiological sensors, and are configured to extract one or more physiological metrics from physiological waveforms. Unfortunately, inaccurate physiological metric extraction can reduce the accuracy of health, fitness and/or vital status monitoring.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Various embodiments described herein can provide physiological signal processing systems for physiological waveforms that include cardiovascular signal components therein. The physiological signal processing system includes a physiological sensor that is configured to generate a physiological waveform that includes a cardiovascular signal component and a noise component therein. A noise reference sensor is configured to generate a noise reference waveform including the noise component therein. A first variable high pass filter is responsive to the physiological waveform and is configured to high pass filter the physiological waveform in response to a first corner frequency that is applied thereto. A second variable high pass filter is responsive to the noise reference waveform and is configured to high pass filter the noise reference waveform in response to a second corner frequency that is applied thereto. A heart rate metric extractor is responsive to the first and second variable high pass filters and is configured to extract a heart rate metric from the physiological waveform that is high pass filtered by the first variable high pass filter and from the noise reference waveform that is high pass filtered by the second variable high pass filter. A corner frequency adjustor is responsive to the heart rate metric extractor and is configured to determine the first and second corner frequencies that are applied to the first and second variable high pass filters, respectively, based on the heart rate metric that was extracted. The first and second corner frequencies may be substantially the same in some embodiments but may be substantially different in other embodiments. A physiological metric assessor may also be provided that is responsive to the heart rate metric extractor and that is configured to process the heart rate metric to generate at least one physiological assessment.

In some embodiments, the noise component comprises a motion component and the noise reference sensor comprises an inertial sensor. The inertial sensor may comprise an accelerometer, a pressure sensor, a blocked channel sensor and/or the like. In some embodiments, the noise reference waveform is substantially devoid of the cardiovascular signal component.

In some embodiments, the heart rate metric extractor is configured to obtain a difference between the physiological waveform that is high pass filtered by the first variable high pass filter and the noise reference waveform that is high pass filtered by the second variable high pass filter. In some embodiments, the heart rate metric extractor comprises a spectral subtractor that is configured to obtain a difference between a frequency domain representation of the physiological waveform that is high pass filtered by the first variable high pass filter and a frequency domain representation of the noise reference waveform that is high pass filtered by the second variable high pass filter.

Physiological waveforms may be processed according to various embodiments described herein. For example, the physiological waveform may include an electroencephalogram (EEG), an electrocardiogram (ECG) and/or a radio frequency (RF) waveform, an electro-optical physiological waveform including a photoplethysmograph (PPG) waveform, an electro-photoacoustic waveform including a photoacoustic waveform, an electro-mechanical physiological waveform including an auscultation waveform, a piezo sensor waveform and/or an accelerometer waveform, and/or an electro-nuclear physiological waveform. Moreover, various physiological assessments may be provided including ventilator threshold, lactate threshold, cardiopulmonary status, neurological status, aerobic capacity (VO₂ max) and/or overall health or fitness.

Various configurations of the first and second variable high pass filters may also be provided according to various embodiments described herein. For example, the first and second variable high pass filters may each comprise a single high pass filter having an adjustable corner frequency, wherein the corner frequency adjustor is configured to determine the adjustable corner frequency. Alternatively, the first and second variable high pass filters may each comprise a plurality of high pass filters, a respective one of which includes a different value of the corner frequency, wherein the corner frequency adjustor is configured to select one of the plurality of high pass filters that corresponds to the corner frequency that is determined.

Various other embodiments of the first and second variable high pass filters may also be provided. Analog variable high pass filters may be provided with adjustable component values thereof. Alternatively, the first and second variable high pass filters may each comprise a variable digital high pass filter having a plurality of delay taps, wherein the corner frequency corresponds to a number of the plurality of delay taps that are selected to filter the physiological waveform. In these embodiments, the corner frequency adjuster may comprise a mapping system that is configured to map the heart rate metric that is extracted from the physiological waveform that is filtered into the number of the delay taps that are selected to high pass filter the physiological waveform.

Various embodiments described herein can also configure the corner frequency adjuster to reduce or prevent locking on an erroneous heart rate metric. In some embodiments, the corner frequency adjuster is configured to initially set predetermined first and second corner frequencies corresponding to a predetermined heart rate prior to determining the first and second corner frequencies that are applied to the first and second variable high pass filters from the heart rate metric. The predetermined heart rate may be a resting heart rate, such as 72 beats per minute. The corner frequency adjuster may also be configured to initially set the predetermined first and second corner frequencies corresponding to the predetermined heart rate until the heart rate metric extractor locks on a heart rate of the physiological waveform. Moreover, the corner frequency adjuster may also be configured to reset or reapply the predetermined first and second corner frequencies corresponding to the predetermined heart rate in response to determining that the physiological sensor is no longer responsive to a source of the physiological waveform. The corner frequency adjuster may also be configured to determine the first and second corner frequencies that are applied to the first and second variable high pass filters from the heart rate metric by applying a margin to the heart rate metric. Moreover, the first and second variable high pass filters may each include a gradual filter transition band (i.e., it is not a brick wall filter).

Various embodiments described herein may also provide physiological signal processing systems that may be used with physiological sensors that are configured to generate a physiological waveform that includes cardiovascular and pulmonary signal components therein. A variable low pass filter is added that is responsive to the physiological waveform and that is configured to low pass filter the physiological waveform in response to a third corner frequency that is applied thereto. A respiration rate metric extractor is provided that is responsive to the variable low pass filter and that is configured to extract a respiration rate metric from the physiological waveform that is filtered by the variable low pass filter. The corner frequency adjustor is further configured to determine the third corner frequency that is applied to the variable low pass filter from the heart rate metric. The first variable high pass filter, the variable low pass filter and/or the heart rate metric extractor may be configured according to any of the filter components described above.

In any of the embodiments described herein, the corner frequency adjuster may include hysteresis to reduce or prevent excessive filter adjustment. Moreover, in any of these embodiments, the at least one corner frequency may comprise substantially the same corner frequency that is applied to the variable high pass and low pass filters.

In any of the embodiments described herein, the sensor may be a plethysmograph sensor and, more specifically, a photoplethysmograph (PPG) sensor and the noise reference sensor may be an accelerometer. Specifically, the physiological signal processing system may comprise a PPG sensor that is configured to generate a PPG waveform that includes a cardiovascular signal component and an acceleration signal component therein, and an accelerometer that is configured to generate an accelerometer waveform including the acceleration signal component therein. A first variable high pass filter is responsive to the PPG waveform and is configured to high pass filter the PPG waveform in response to a first corner frequency that is applied thereto. A second variable high pass filter is responsive to the accelerometer waveform and is configured to high pass filter the accelerometer waveform in response to a second corner frequency that is applied thereto. A heart rate metric extractor is responsive to the first and second variable high pass filters and is configured to extract a heart rate metric from the PPG waveform that is high pass filtered by the first variable high pass filter and from the accelerometer waveform that is high pass filtered by the second variable high pass filter. A corner frequency adjustor is responsive to the heart rate metric extractor and is configured to determine the first and second corner frequencies that are applied to the first and second variable high pass filters, respectively, based on the heart rate metric that was extracted.

Various embodiments have been described above in connection with physiological signal processing systems. However, analogous physiological signal processing methods may also be provided according to various embodiments described herein. For example, some embodiments described herein can provide a physiological signal processing method for a physiological waveform that includes a cardiovascular signal component and a noise component therein, and a noise reference waveform including the noise component therein. The physiological signal processing method comprises high pass filtering the physiological waveform in response to a first adjustable high pass filter corner frequency, high pass filtering the noise reference waveform in response to a second adjustable high pass filter corner frequency, extracting a heart rate metric from the physiological waveform that is high pass filtered and from the noise reference waveform that is high pass filtered and determining the first and second adjustable high pass filter corner frequencies from the heart rate metric that was extracted. Other embodiments corresponding to the above described system embodiments may also be provided.

### BRIEF DESCRIPTION OF THE DRAWINGS

FIGS. 1-4 are functional block diagrams of physiological signal processing systems and methods according to various embodiments described herein.

FIG. 5 is a functional block diagram of a digital variable high pass filter according to various embodiments described herein.

FIG. 6 is a functional block diagram of a digital variable low pass filter according to various embodiments described herein.

FIG. 7 is a flowchart of operations that may be performed by a corner frequency adjuster according to various embodiments described herein.

FIG. 8 graphically illustrates adjusting a corner frequency of a variable high pass filter according to various embodiments described herein.

FIG. 9 graphically illustrates adjusting a corner frequency of a variable low pass filter according to various embodiments described herein.

FIGS. 10-15 illustrate measured waveforms according to various embodiments described herein.

FIGS. 16-18 are functional block diagrams of physiological signal processing systems and methods according to various embodiments described herein.

### DETAILED DESCRIPTION

The present invention will now be described more fully hereinafter with reference to the accompanying figures, in which various embodiments are shown. This invention may, however, be embodied in many different forms and should not be construed as limited to the embodiments set forth herein. Like numbers refer to like elements throughout. The sequence of operations (or steps) is not limited to the order presented in the figures and/or claims unless specifically indicated otherwise. Features described with respect to one figure or embodiment can be associated with another embodiment or figure although not specifically described or shown as such.

It will be understood that, when a feature or element is referred to as being “connected”, “attached”, “coupled” or “responsive” to another feature or element, it can be directly connected, attached, coupled or responsive to the other feature or element or intervening features or elements may be present. In contrast, when a feature or element is referred to as being “directly connected”, “directly attached”, “directly coupled” or “directly responsive” to another feature or element, there are no intervening features or elements present.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

It will be understood that although the terms first and second are used herein to describe various features/elements, these features/elements should not be limited by these terms. These terms are only used to distinguish one feature/element from another feature/element. Thus, a first feature/element discussed below could be termed a second feature/element, and similarly, a second feature/element discussed below could be termed a first feature/element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “headset” includes any type of device or earpiece that may be attached to or near the ear (or ears) of a user and may have various configurations, without limitation. Headsets as described herein may include mono headsets (one earbud) and stereo headsets (two earbuds), earbuds, hearing aids, ear jewelry, face masks, headbands, and the like.

The term “real-time” is used to describe a process of sensing, processing, or transmitting information in a time frame which is equal to or shorter than the minimum timescale at which the information is needed. For example, the real-time monitoring of pulse rate may result in a single average pulse-rate measurement every minute, averaged over 30 seconds, because an instantaneous pulse rate is often useless to the end user. Typically, averaged physiological and environmental information is more relevant than instantaneous changes. Thus, in the context of embodiments of the present invention, signals may sometimes be processed over several seconds, or even minutes, in order to generate a “real-time” response.

The term “monitoring” refers to the act of measuring, quantifying, qualifying, estimating, sensing, calculating, interpolating, extrapolating, inferring, deducing, or any combination of these actions. More generally, “monitoring” refers to a way of getting information via one or more sensing elements. For example, “blood health monitoring” includes monitoring blood gas levels, blood hydration, and metabolite/electrolyte levels.

The term “physiological” refers to matter or energy of or from the body of a creature (e.g., humans, animals, etc.). In embodiments of the present invention, the term “physiological” is intended to be used broadly, covering both physical and psychological matter and energy of or from the body of a creature. However, in some cases, the term “psychological” is called-out separately to emphasize aspects of physiology that are more closely tied to conscious or subconscious brain activity rather than the activity of other organs, tissues, or cells.

The term “body” refers to the body of a subject (human or animal) who may wear a headset incorporating embodiments of the present invention.

In the included figures, various embodiments will be illustrated and described. However, it is to be understood that embodiments of the present invention are not limited to those worn by humans.

The ear is an ideal location for wearable health and environmental monitors. The ear is a relatively immobile platform that does not obstruct a person's movement or vision. Headsets located at an ear have, for example, access to the inner-ear canal and tympanic membrane (for measuring core body temperature), muscle tissue (for monitoring muscle tension), the pinna and earlobe (for monitoring blood gas levels), the region behind the ear (for measuring skin temperature and galvanic skin response), and the internal carotid artery (for measuring cardiopulmonary functioning), etc. The ear is also at or near the point of exposure to: environmental breathable toxicants of interest (volatile organic compounds, pollution, etc.); noise pollution experienced by the ear; and lighting conditions for the eye. Furthermore, as the ear canal is naturally designed for transmitting acoustical energy, the ear provides a good location for monitoring internal sounds, such as heartbeat, breathing rate, and mouth motion.

Wireless, Bluetooth®-enabled, and/or other personal communication headsets may be configured to incorporate physiological and/or environmental sensors, according to some embodiments of the present invention. As a specific example, Bluetooth® headsets are typically lightweight, unobtrusive devices that have become widely accepted socially. Moreover, Bluetooth® headsets are cost effective, easy to use, and are often worn by users for most of their waking hours while attending or waiting for cell phone calls. Bluetooth® headsets configured according to embodiments of the present invention are advantageous because they provide a function for the user beyond health monitoring, such as personal communication and multimedia applications, thereby encouraging user compliance. Exemplary physiological and environmental sensors that may be incorporated into a Bluetooth® or other type of headsets include, but are not limited to accelerometers, auscultatory sensors, pressure sensors, humidity sensors, color sensors, light intensity sensors, pressure sensors, etc.

Optical coupling into the blood vessels of the ear may vary between individuals. As used herein, the term “coupling” refers to the interaction or communication between excitation light entering a region and the region itself. For example, one form of optical coupling may be the interaction between excitation light generated from within a light-guiding earbud and the blood vessels of the ear. Light guiding earbuds are described in co-pending U.S. Patent Application Publication No. 2010/0217102, which is incorporated herein by reference in its entirety. In one embodiment, this interaction may involve excitation light entering the ear region and scattering from a blood vessel in the ear such that the intensity of scattered light is proportional to blood flow within the blood vessel. Another form of optical coupling may be the interaction between excitation light generated by an optical emitter within an earbud and the light-guiding region of the earbud.

Various embodiments described herein are not limited to headsets that communicate wirelessly. In some embodiments of the present invention, headsets configured to monitor an individual's physiology and/or environment may be wired to a device that stores and/or processes data. In some embodiments, this information may be stored on the headset itself. Furthermore, various embodiments described herein are not limited to earbuds. Some embodiments may be employed around another part of the body, such as a digit, finger, toe, limb, wrist, around the nose or earlobe, or the like. Other embodiments may be integrated into a patch, such as a bandage that sticks on a person's body.

The specification that follows will first describe various embodiments described in application Ser. No. 14/124,465 to the present inventor, Eric Romesburg, entitled “Systems and Methods for Variable Filter Adjustment by Heart Rate Metric Feedback”, assigned to the Assignee of the present application, the disclosure of which is hereby incorporated herein by reference in its entirety. Then, a new section entitled “Systems and Methods for Variable Filter Adjustment by Heart Rate Metric Feedback and Noise Reference Sensor,” will be provided.

### Systems and Methods for Variable Filter Adjustment by Heart Rate Metric Feedback

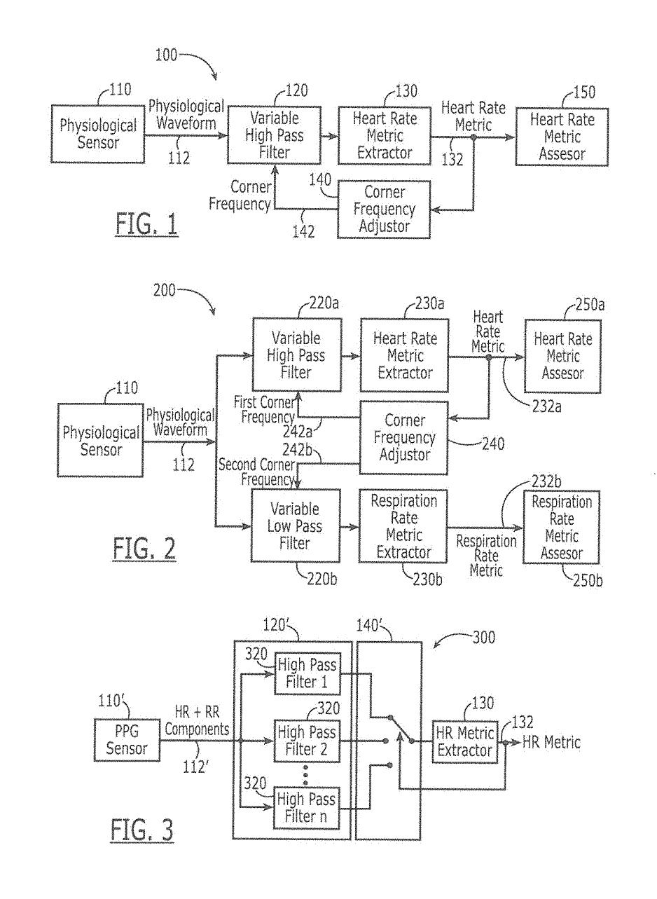

This section of the specification and FIGS. 1-15 correspond to the above-cited application Ser. No. 14/124,465.

Various embodiments described herein may arise from recognition that a physiological signal component in a physiological waveform may change dramatically over time, for example due to the user's activity level and/or other factors. In order to effectively extract a physiological metric from the physiological waveform, the physiological metric itself may be used to directly or indirectly adjust a parameter of a variable filter, such as a filter's low pass or high pass corner frequency. Accordingly, accurate filtering may be provided and accurate parameter extraction may be obtained, notwithstanding the large changes that may take place in the value of the physiological metric.

It also may be exceedingly difficult to extract metrics from physiological sensors that generate physiological waveforms that include multiple physiological signal components therein. For example, a physiological sensor, such as a plethysmograph or a photoplethysmograph, may include cardiovascular and pulmonary signal components therein. Unfortunately, these physiological metrics have overlapping frequency ranges. For example, the cardiovascular signal component (heart rate) may range from about 45 beats per minute to about 220 beats per minute, while the pulmonary signal component (respiration rate) may range from about 12 breaths per minute to about 70 breaths per minute. Due to the overlap, it may be exceedingly difficult to separate the two physiological components.

However, various embodiments described herein may arise from further recognition that, in general, although heart rate and respiration rate may overlap, their rise and fall may generally track due to, for example, changes in physical activity or the environment. Thus, they may both generally go up together and go down together. Accordingly, various embodiments described herein can provide a variable high pass and a variable low pass filter having at least one corner frequency that can be varied in response to a heart rate metric that is extracted from the high pass filtered physiological waveform. By providing variable filter adjustment using physiological metric feedback, the heart and/or respiration rate may be extracted accurately, notwithstanding the fact that they are contained in the same signal and overlap in their frequency ranges.

Various embodiments described herein may also arise from recognition that it did not appear to be heretofore possible to use an extracted heart rate to control a high pass filter that feeds a heart rate metric extractor. Specifically, due to the possibility for the extracted heart rate to be in error, the high pass filter may blind the metric extractor from the heart rate frequency in the physiological waveform signal. In other words, the heart rate metric extractor may get stuck at a high rate and, due to the high pass filtering that takes place, may never become responsive to the heart rate in the physiological waveform. Accordingly, the heart rate metric extractor may diverge or run away from the actual heart rate. Yet, despite these potential problems, various embodiments described herein can allow an extracted heart rate metric to be used to set a variable high pass filter corner frequency, and in some embodiments to also set a variable low pass filter corner frequency, while reducing or eliminating the heart rate extractor from being blinded to its own frequency.

Accordingly, various embodiments described herein can reduce or prevent locking on an erroneous heart rate metric. Thus, a heart rate metric can be used to set a corner frequency of a variable high pass filter for heart rate extraction. Moreover, the heart rate metric that is extracted may also be used to set a corner frequency for a variable low pass filter for respiration rate extraction, according to various embodiments described herein.

FIG. 1 is a functional block diagram of physiological signal processing systems and methods according to various embodiments described herein. Referring now to FIG. 1, these physiological signal processing systems/methods 100 may be used to process a physiological waveform 112 that is produced by a physiological sensor 110, and that includes a physiological signal component therein. The physiological waveform 112 may include an electrical physiological waveform including an electroencephalogram (EEG), an electrocardiogram (ECG) and/or a radio frequency (RF) waveform, an electro-optical physiological waveform including a photoplethysmograph (PPG) waveform, an electro-photoacoustic waveform including a photoacoustic waveform, an electro-mechanical physiological waveform including an auscultation waveform, a piezo sensor waveform and/or an accelerometer waveform, and/or an electro-nuclear physiological waveform. The physiological signal component may include a neurological, cardiovascular and/or pulmonary signal component. For example, in some embodiments, the physiological sensor 110 may be a plethysmograph sensor, such as a photoplethysmograph (PPG) sensor, and the physiological waveform may include both cardiovascular and pulmonary signal components therein.

Still referring to FIG. 1, a heart rate metric extractor 130 extracts a heart rate metric 132 from the physiological waveform 112. The heart rate metric extractor 130 may extract the heart rate metric using one or more conventional techniques. Moreover, a heart rate metric assessor 150 may be provided to assess the heart rate metric according to one or many known physiological metric assessment techniques. The physiological assessment may include ventilator threshold, lactate threshold, cardiopulmonary status, neurological status, aerobic capacity (VO₂ max) and/or overall health or fitness.

Still referring to FIG. 1, the heart rate metric extractor 130 is coupled to the physiological sensor 110 by a variable high pass filter 120. The variable high pass filter 120 is responsive to the physiological sensor 110, and is configured to high pass filter the physiological waveform 112 in response to a corner frequency 142 that is applied thereto.

The high pass filter 120 may include a single analog or digital high pass filter having an adjustable corner frequency 142. Alternatively, the variable high pass filter 120 may comprise a plurality of analog or digital high pass filters, a respective one of which includes a different value of the corner frequency 142. Moreover, depending on the physiological waveform that is processed, the variable filter may be a variable high pass, low pass, bandpass, notch and/or other filter, and the filter parameter may be a low pass filter corner frequency, a high pass filter corner frequency, a bandpass filter corner frequency and/or bandwidth and/or a notch frequency. The variable digital filter may be embodied by a plurality of delay taps, the number of which is selected to provide the variable filtering.

Still continuing with the description of FIG. 1, a corner frequency adjuster 140 is provided that is responsive to the heart rate metric extractor 130 and is configured to determine the corner frequency 142 that is applied to the variable high pass filter 120 based on the heart rate metric 132 that was extracted. Accordingly, variable filter adjustment by physiological metric feedback is provided.

Many embodiments of corner frequency adjusters 140 will be described in detail below. In general, the corner frequency adjuster 140 may be configured to determine a corner frequency that is applied to the variable high pass filter 120 or to select from among a plurality of variable high pass filters, for example by selecting a number of delay taps in a variable digital high pass filter. For example, as will be described in more detail below, the corner frequency adjuster 140 may include a mapping system that is configured to map the heart rate metric 132 that is extracted from the physiological waveform 112 that is filtered by the variable high pass filter 120, into a number of delay taps that is selected to filter the physiological waveform 112 by the variable high pass filter 120.

FIG. 2 is a functional block diagram of physiological signal processing systems and methods according to various other embodiments described herein. These physiological signal processing systems and methods 200 are configured to extract cardiovascular and pulmonary physiological signal components that are included in a physiological waveform 112 as provided by the physiological sensor 110. In some embodiments, the cardiovascular and pulmonary physiological signal components rise and fall in frequency roughly in tandem, and the cardiovascular signal component includes a highest frequency that is higher than the lowest frequency of the pulmonary signal component. It will be understood that more than two physiological signal components may be processed in other embodiments, but only two components are illustrated in FIG. 2 for ease of illustration.

In embodiments of FIG. 2, a variable high pass filter 220a and a variable low pass filter 220b may be provided. The variable high pass filter 220a is responsive to the physiological waveform 112 and is configured to high pass filter the physiological waveform in response to a first corner frequency 242a that is applied thereto. The variable low pass filter 220b is responsive to the physiological waveform 112 and is configured to low pass filter the physiological waveform in response to a second corner frequency 242b that is applied thereto. The first and second corner frequencies 242a, 242b may be identical in some embodiments, and may be different in other embodiments. Moreover, when the first physiological signal component is a cardiovascular signal component and the second physiological signal component is a pulmonary signal component, the first corner frequency 242a is a high pass corner frequency and the second corner frequency 242b is a low pass corner frequency.

Continuing with the description of FIG. 2, a heart rate metric extractor 230a and a respiration rate metric extractor 230b may be provided. The heart rate metric 232a may be processed and analyzed by a heart rate metric assessor 250a, and the respiration rate 232b may be processed and analyzed by a respiration rate metric assessor 250b. Many techniques for operating heart rate and respiration rate metric extractors and assessors are known, and need not be described further herein.

Still referring to FIG. 2, a corner frequency adjuster 240 is provided. The corner frequency adjuster 240 is responsive to the heart rate metric extractor 230a to determine the first and second corner frequencies 242a and 242b that are applied to the variable high pass and low pass filters 220a and 220b, respectively. Various embodiments of the corner frequency adjuster 240 may be provided. Various detailed examples will be provided below.

FIG. 3 is a functional block diagram of an embodiment 300 of FIG. 1 that may be used to extract a heart rate metric 132 from the physiological waveform 112′ that is produced by a PPG sensor 110′, wherein the physiological waveform 112′ includes both heart rate (HR) and respiration rate (RR) components. A variable high pass filter 120′ is embodied in FIG. 3 by a plurality of high pass filters 320, each of which includes a different corner frequency. The heart rate metric extractor 130 is configured to extract a heart rate metric 132 using any known technique. The corner frequency adjuster 140 of FIG. 1 is embodied by a corner frequency adjuster 140′, represented as a switch that is configured to select one of the plurality of high pass filters 320 that corresponds to the corner frequency that is determined. For example, in some embodiments, the corner frequency adjuster 140′ uses a mapping function to select one of the plurality of high pass filters 320 that has a corner frequency that is within a margin of the heart rate metric 132′. In some embodiments, the margin may correspond to a margin of between about 18 and about 30 beats per minute below the heart rate metric 132′. By selecting the appropriate high pass filter 320, the corner frequency adjuster 140′ can reduce or prevent the respiration rate component from interfering with the extraction of the heart rate component.

It will be understood that the margin may be selected as a function of the heart rate metric 132. For example, a table lookup may be used to map a heart rate metric 132 that is extracted into a desired high pass filter corner frequency, and then the filter 320 may be selected that has a corner frequency that is closest to the mapped corner frequency. It will also be understood that hysteresis may be used to reduce or prevent switching of the high pass filters 320 too rapidly, because the rapid switching may adversely affect the extraction of the heart rate metric by the heart rate metric extractor 130.

In other embodiments of FIG. 3, it may also be desirable to extract a respiration rate metric from the waveform 112′, so that low pass filters may be used in addition to high pass filters 320. The corner frequency adjuster 140′ may be configured to adjust low pass filter corner frequency by applying a given margin below the heart rate metric.

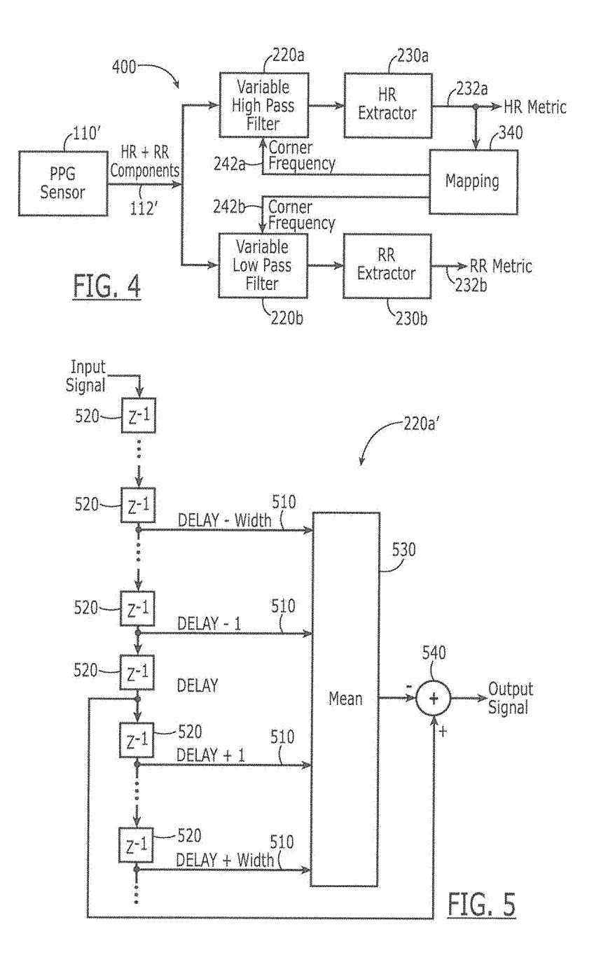

FIG. 4 is a more detailed functional block diagram of embodiments 400 of FIG. 2, and may be used to extract a heart rate metric 232a and a respiration rate metric 232b from a PPG sensor 110′ that provides a PPG sensor waveform 112′ that includes both heart rate and respiration rate components. A variable high pass filter 220a and a variable low pass filter 220b is provided. Each of these filters may be embodied by a single filter with an adjustable corner frequency or by multiple filters having different corner frequencies, one of which may be selected. Heart rate extractor 230a and respiration rate extractor 230b are responsive to the variable high pass filter 220a and the variable low pass filter 220b, respectively, so as to obtain a heart rate metric 232a and a respiration rate metric 232b. The corner frequency adjuster previously described may be embodied by a mapping function 340. As shown in embodiments of FIG. 4, the mapping function 340 is responsive to the heart rate metric 232a and is responsible for determining both the high pass filter corner frequency 242a and the low pass filter corner frequency 242b. In some embodiments, the same corner frequency may be used for both the variable high pass filter 220a and the variable low pass filter 220b. In other embodiments, the mapping function 340 may determine different corner frequencies 242a and 242b.

In embodiments of FIG. 4, only the heart rate metric 232a is used by the mapping function to determine the corner frequency for both the variable high pass filter 220a and the variable low pass filter 220b. It has been found, according to various embodiments described herein, that the heart rate metric 232a may provide a more accurate basis for determining both corner frequencies, because the heart rate metric may be subject to less conscious control by the user compared to the respiration rate metric. A specific mapping function will be described below.

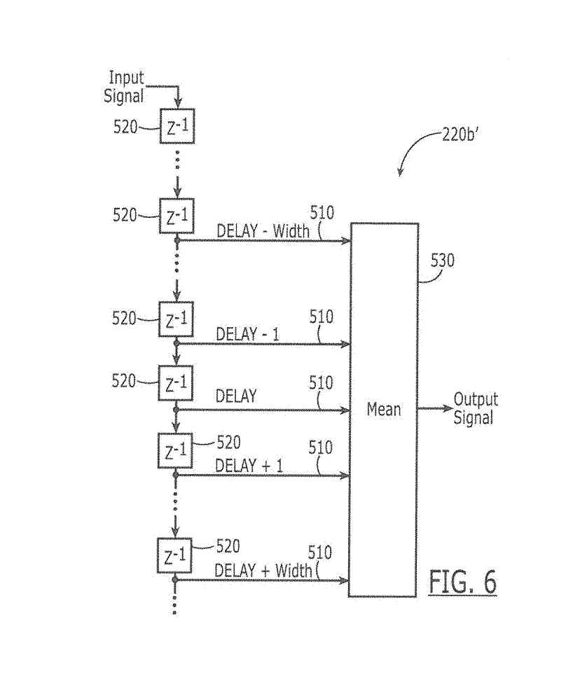

As was described above, the variable high pass filter 220a and/or the variable low pass filter 220b of FIG. 4 may comprise a variable digital high pass filter and/or a variable digital low pass filter. FIG. 5 illustrates an embodiment of a variable digital high pass filter, and FIG. 6 illustrates an embodiment of a variable digital low pass filter.

Referring to FIG. 5, these embodiments of a variable high pass filter 220a′ include a plurality of high pass delay taps 510 that are generated by a plurality of digital delay elements 520. The digital delay elements 520 are responsive to an input signal, which may be the physiological waveform 112′ of a PPG sensor, and the variable high pass filter 220a′ provides an output signal, which may be provided to a metric extractor, such as an HR extractor 230a of FIG. 4. A “width” parameter determines the number of delay taps that are selected by a mean block 530 and a summing node 540, to determine the output signal. As shown in FIG. 6, the variable low pass filter 220b′ can include a similar architecture. However, a summer 540 may not be needed in the variable low pass filter 220b′ because the “DELAY” tap already feeds the mean block 530 with the same sign as the other taps 510.

Accordingly, FIGS. 5 and 6 illustrate various embodiments wherein the variable high pass filter (FIG. 5) and/or the variable low pass filter (FIG. 6) comprises a variable digital high pass filter 220a′ and/or a variable digital low pass filter 220b′ having a plurality of high pass and/or low pass delays taps 510, respectively, wherein the corner frequency is determined by a number of high pass and/or low pass delay taps 510 that are selected to filter the physiological waveform.

A specific embodiment of a mapping function 340 will now be described. In these embodiments, the mapping function 340 is configured to determine a corner frequency 242a of the variable high pass filter 220a′ and the corner frequency 242b of the variable low pass filter 220b′ by applying a margin to the heart rate metric 232a, and is further configured to determine the number of delay taps 510 from the corner frequency that was determined.

A mathematical description of this mapping function 340 may be provided by Equations (1) and (2):

CornerFreq=max(MINIMUM_HR_BPM,HeartRate−MARGIN_BPM) (1)

Width=round(DELAY*MINIMUM_HR_BPM/CornerFreq) (2)

In Equations (1) and (2), variables in CAPITAL_LETTERS are predetermined constants, while variables in CamelCase may change every frame. In this mapping function, CornerFreq is the corner frequency 242a and 242b. MINIMUM_HR_BPM is the minimum heart rate to be measured in beats per minute. HeartRate is the heart rate metric 232a that is measured. MARGIN_BPM is a desired margin between the reported heart rate and the corner frequency of the variable filter, which may be empirically determined. The margin allows for some error in the reported heart rate without causing significant attenuation by the variable high pass filter. Accordingly, in Equation (1) the corner frequency is determined by the maximum of either the minimum heart rate or the measured heart rate minus the margin that is set. Moreover, in Equation (2), Width is the parameter in FIGS. 5 and 6 that determines the number of delay taps 510. Specifically, in FIGS. 5 and 6, two times the width determines the number of delay taps that are input into the mean block 530. As shown in Equation (2), the width may be determined by rounding up or down the value of the delay of each of the delay elements 520 multiplied by the minimum heart rate divided by the corner frequency that was determined in Equation (1).

Accordingly, Equations (1) and (2) illustrate an embodiment wherein the mapping function 340 is configured to determine a corner frequency of the variable low pass filter 220b and the variable high pass filter 220a by determining a maximum of a minimum heart rate, and the heart rate metric 232a minus the margin, and is further configured to determine the number of delay taps 510 by rounding a product of the delay 520 of the delay taps 510 and the minimum heart rate divided by the corner frequency 242a/242b that was determined. It will be understood, however, that many other mapping functions may be provided according to other embodiments described herein.

Embodiments that were described above in connection with FIGS. 1-4 use a heart rate metric that was extracted to provide corner frequency adjustment of a variable high pass filter for a heart rate metric extractor, and may also use the heart rate metric that was extracted to adjust a corner frequency of a variable low pass filter of a respiration rate metric extractor. Heretofore, it does not appear that feedback of an extracted heart rate was used to control a high pass filter feeding a heart rate metric extractor. Specifically, because of the possibility for the extracted heart rate to be in error, the variable high pass filter may blind the heart rate metric extractor from the heart rate frequency in the physiological waveform. Stated differently, the heart rate metric extractor could lock on, i.e., get stuck at, a high rate, and remain unresponsive to the actual heart rate in the physiological waveform. Specifically, if the heart rate metric extractor locks on a high rate, the variable high pass filter may filter out the actual (lower) heart rate frequency in the physiological waveform. Accordingly, the heart rate metric extractor may diverge or run away from the actual heart rate. Thus, heretofore, feedback of the extracted heart rate to control the high pass filter feeding the heart rate metric extractor does not appear to have been used. In sharp contrast, various embodiments described herein can reduce or prevent locking on an erroneous heart rate metric. Thus, various embodiments described herein can use feedback of the heart rate metric that was extracted to control the high pass filter feeding the heart rate metric extractor, as was illustrated in FIGS. 1-4. The heart rate metric that was extracted may also be used to feed the low pass filter for the respiration rate metric extractor, as was shown in FIGS. 2 and 4.

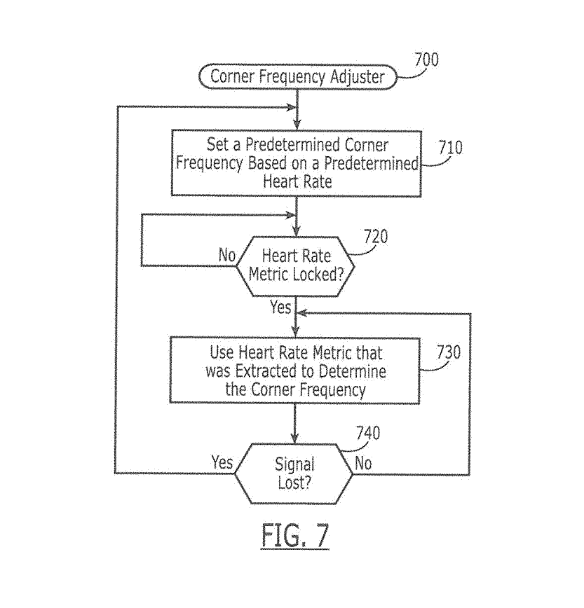

FIG. 7 is a flowchart of operations that may be performed by a corner frequency adjuster 700, such as the corner frequency adjuster 140 of FIG. 1, the corner frequency adjuster 240 of FIG. 2, the corner frequency adjuster 140′ of FIG. 3, or the mapping function 340 of FIG. 4, to reduce or prevent an erroneous extracted heart rate from blinding the heart rate metric extractor to the heart rate frequency in the physiological waveform. Referring now to FIG. 7, the corner frequency adjuster 700 may be configured to initially set at least one predetermined corner frequency corresponding to a predetermined heart rate prior to determining the at least one corner frequency that is applied to the variable high pass filter, and in some embodiments to the variable low pass filter, from the heart rate metric, as illustrated at Block 710. Thus, at startup, the extracted heart rate 132 may be initialized to a predetermined, relatively low heart rate, such as a resting heart rate of, for example, 72 beats per minute. By initially setting the extracted heart 132 at a low value, subsequent blinding of the metric extractor by the high pass filter that is set for a high heart rate, may be reduced or prevented. Thus, the corner frequency adjuster 700 is configured to reduce or prevent locking on an erroneous heart rate in the physiological waveform.

Then, at Block 720, once a heart rate metric is locked, the heart rate metric that was extracted may be used to determine the corner frequency at Block 730. Thus, Blocks 710-730 illustrate the use of a “hunting mode” at startup, where the corner frequency of the high pass filter, and in some embodiments of the low pass filter, is set at a predetermined frequency (Block 710) until the heart rate metric extractor locks on the heart rate PPG signal at Block 720. Then, the heart rate metric that was extracted may be used at Block 730.

One way to determine that the heart rate metric extractor has locked on the heart rate in the physiological waveform at Block 720 is to determine when the physiological waveform spectral peak is within a window around the extracted heart rate. The window may be a predetermined window that remains constant, or may be a variable window. If the spectral peak is within the window around the extracted heart rate, the heart rate may be deemed to be believed, whereas if it is outside the window, it could be noise, and therefore be erroneous.

Finally, at Block 740, a determination may be made that the physiological waveform signal is lost, for example, because the physiological sensor 110 goes off the body. A determination that the physiological sensor goes off the body may be obtained using a proximity sensor and/or other techniques. If the signal is lost at Block 740, operations may return to Block 710 to reset (i.e., reapply) the predetermined heart rate and then return into hunting mode at Blocks 720 and 730. On the other hand, as long as the signal is not lost at Block 740, the heart rate metric that was extracted may continue to be used to determine the at least one corner frequency at Block 730. Thus, the corner frequency adjuster is configured to reduce or prevent locking on an erroneous heart rate metric.

Other techniques may also be used to reduce or prevent the high pass filter from blinding the metric extractor to the heart rate frequency in the physiological waveform. For example, the high pass filters 120, 220a, 120′ or 220a′ may all use a gradual filter transition band. Stated differently, brick wall high pass filters are not used in these embodiments. Thus, the gradual transition high pass filter may have a greater ability to include the heart rate frequency in the high pass filtered signal. Another technique may use a margin between the extracted heart rate and the corner frequency of the high pass filter. For example, 18 beats per minute margin may be used, as was already described. The above described techniques may be used individually, or in various combinations and subcombinations, to reduce or prevent the high pass filter from blinding the metric extractor from the heart rate frequency in the physiological waveform, and thereby reduce or prevent locking on an erroneous heart rate metric.

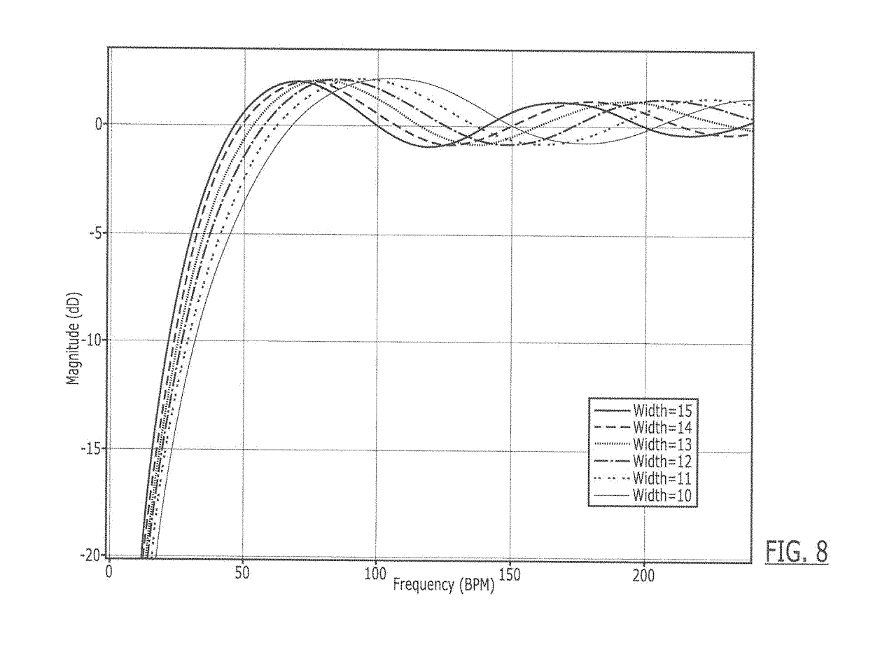

FIG. 8 illustrates how the corner frequency of a variable high pass filter, such as the variable high pass filter 220a′ of FIG. 5, may be varied using the mapping function 340 described by Equations (1) and (2) above, according to various embodiments described herein. FIG. 8 assumes a value of DELAY of 15 samples and plots the frequency response of the variable high pass filter 220a′ with various width values from 10 to 15 at a sample rate of 25 Hz. As shown, the corner frequency of the variable high pass filter 220a′, which may be measured at, for example, the −2 dB, −3 dB or −6 dB magnitude, can be varied by varying the width parameter. The high pass filter of FIG. 8 may be used to extract the heart rate metric 232a.

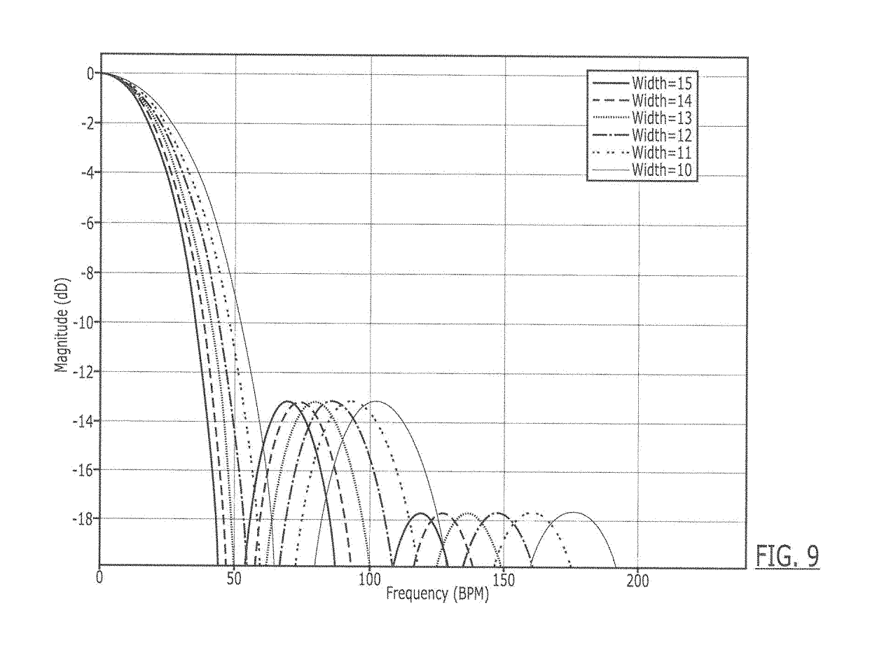

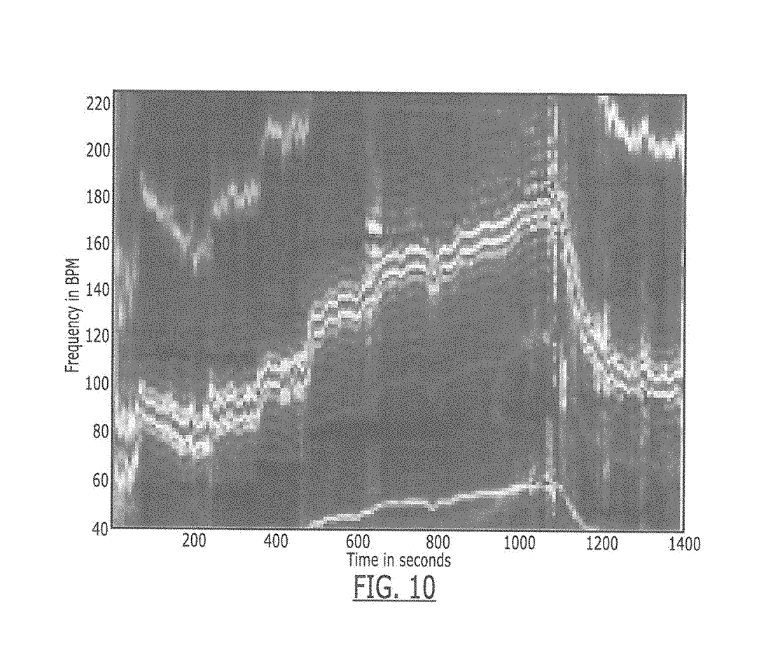

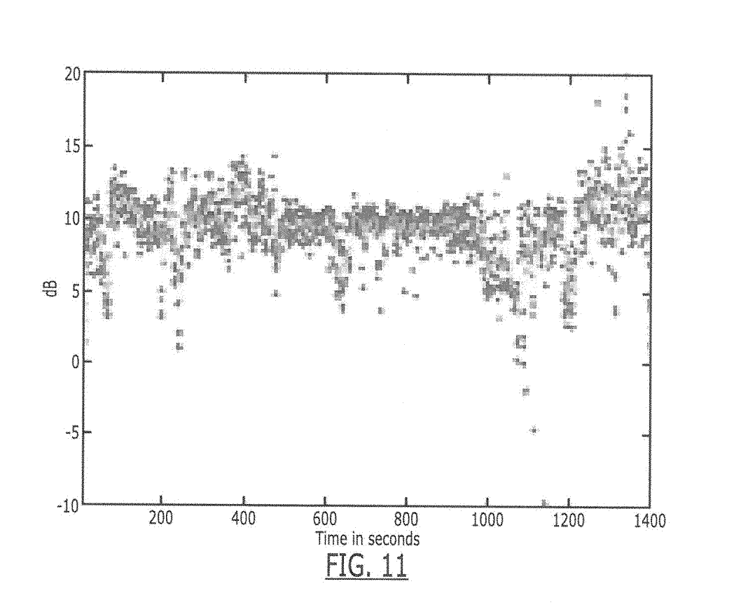

FIG. 9 illustrates analogous operations for a variable digital low pass filter 220b′, such as illustrated in FIG. 6. Again, in FIG. 9, a DELAY value of 15 samples is plotted as a function of widths from 10 to 15. The cutoff frequency may be measured, for example, by the −12 dB, −10 dB or −8 dB points in the magnitude. Accordingly, variable cutoff frequency low pass filters may be used to extract the respiration rate metric 232b.

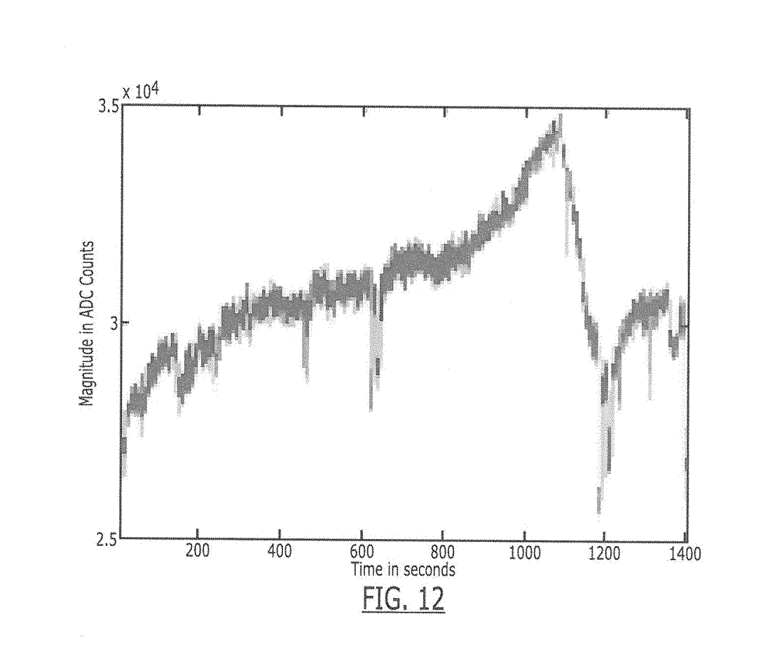

FIGS. 10-15 illustrate other measured results. Specifically, FIG. 10 graphically illustrates a typical noise-reduced PPG spectrum.

FIG. 11 illustrates a signal-to-noise-and-distortion measurement for the same signal in FIG. 10.

FIG. 12 illustrates raw samples of the physiological waveform 112′ that may be obtained by a PPG sensor 110′.

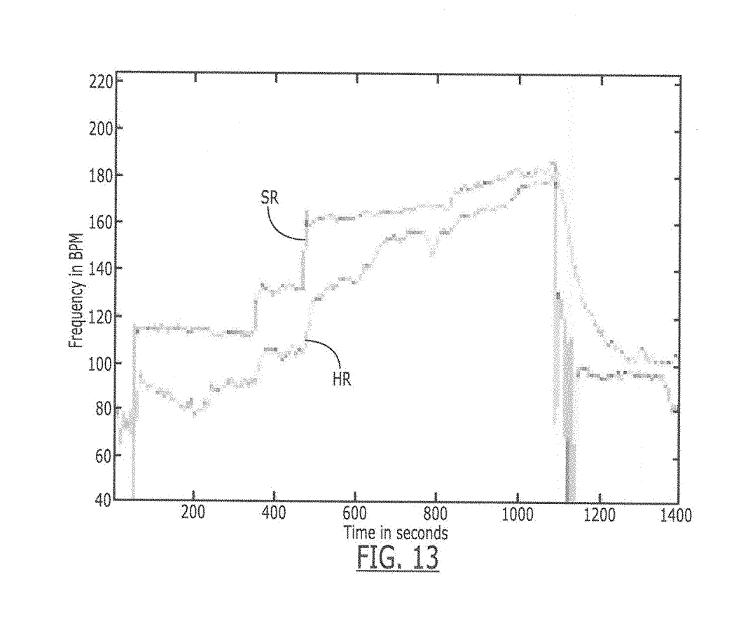

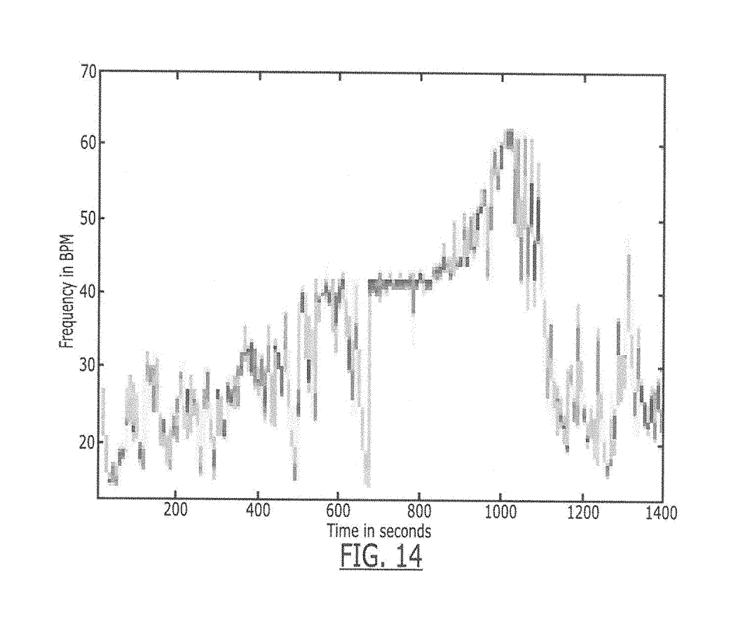

FIG. 13 illustrates heart rate HR (232a of FIG. 4) and step rate SR (in steps per minute) that may be extracted by a system and/or method of FIG. 4 over time.

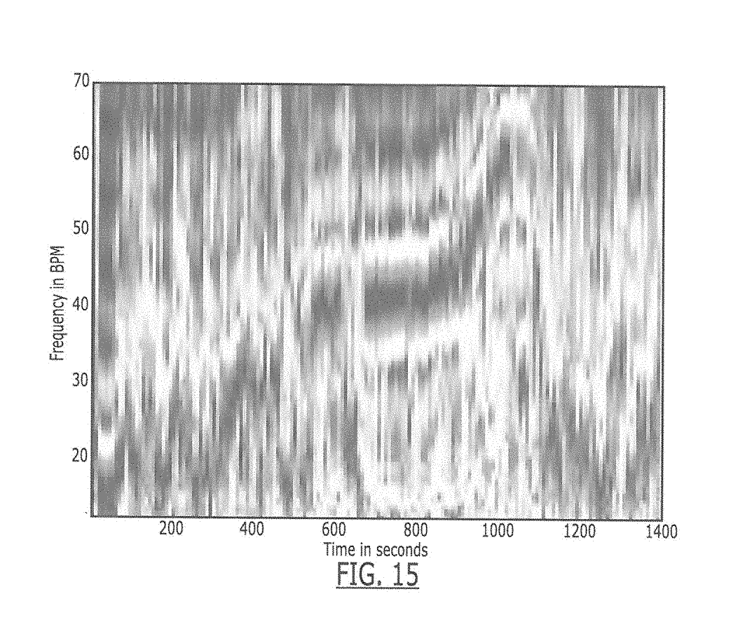

FIG. 14 illustrates a respiration rate metric that may be provided by a respiration rate extractor 230b of FIG. 4 as a function of time. Finally, FIG. 15 illustrates a peak normalized spectrum for the respiration rate.

Various embodiments have been described herein primarily with respect to physiological signal processing systems. However, FIGS. 1-7 also describe analogous physical signal processing methods according to various embodiments described herein. For example, various analogous method embodiments described herein can select among multiple filters for extracting a physiological signal component, where the selection is controlled by an extracted physiological metric. The physiological metric can be the same or different than the physiological component. For example, an extracted heart rate metric can control the filtering of both heart rate and respiration rate. Variable filter adjustment by physiological waveform metric feedback may thereby be provided.

### Systems and Methods for Variable Filter Adjustment by Heart Rate Metric Feedback and Noise Reference Sensor

Various embodiments of systems and methods for variable filter adjustment by heart rate metric feedback and noise reference sensor will now be described. The embodiments that will now be described may arise from a recognition that improved heart rate metric extraction may be obtained, for example relative to systems and methods described in the above cited application Ser. No. 14/124,465 and in FIGS. 1-15 herein that were described above, when a heart rate metric extractor extracts a heart rate metric using a physiological waveform from a physiological sensor and a noise reference waveform from a noise reference sensor. The noise reference sensor can comprise an inertial sensor such as an accelerometer, a pressure sensor, and/or a block channel sensor. The physiological sensor generates a physiological waveform that includes a cardiovascular signal component and a noise component therein. The noise reference sensor is configured to generate a noise reference waveform including the noise component therein. The physiological waveform may be filtered by a first high pass filter and the noise reference waveform from the noise reference sensor may be filtered by a second high pass filter. The two filtered noise reference waveform may then be processed by a heart rate metric extractor to more efficiently extract a heart rate metric in the presence of a noise signal, which may be caused, for example, by user acceleration.

According to various embodiments described herein, the physiological waveform is filtered by a first variable high pass filter that is responsive to a first corner frequency that is applied thereto. A second variable high pass filter is provided that is responsive to the noise reference waveform from the noise reference sensor and that is configured to high pass filter the noise reference waveform in response to a second corner frequency that is applied thereto. The heart rate metric extractor is configured to extract a heart rate metric from the physiological waveform that is high pass filtered by the first variable high pass filter and the noise reference waveform that is high pass filtered by the second variable high pass filter. The corner frequency adjustor is responsive to the heart rate metric adjustor and is configured to determine first and second corner frequencies that are applied to the first and second variable high pass filters, respectively, based on the heart rate metric that was extracted. The first and second corner frequencies may be substantially the same or substantially different.

Accordingly, various embodiments described herein can extract a heart rate metric, and in some embodiments may also extract a respiration rate metric, in the presence of, for example, motion noise. The heart rate metric extraction may be more accurate than when using fixed high pass filters, or when using variable high pass filters without a separate noise reference sensor.

Specifically, although various embodiments described in connection with FIGS. 1-15 may be used to effectively extract a hear rate metric, and in some embodiments a respiration rate metric, from a physiological waveform that is produced by a physiological sensor, it may be exceedingly difficult for these embodiments to extract the heart rate metric/respiration rate metric when the motion noise is stronger than the heart rate component, and also above the variable high pass filter corner frequency. Moreover, even if a noise reference sensor is used with spectral subtraction, the heart rate metric extraction may be most effective if the motion noise is periodic. In contrast, when the motion noise is non-periodic, and thus broadband, various embodiments that will now be described can better match the spectral shapes of the heart rate component and an acceleration signal component that are provided to a spectral subtractor. This can provide greater attenuation of motion noise, which can result in more effective heart rate metric extraction, even when the motion noise is non-periodic.

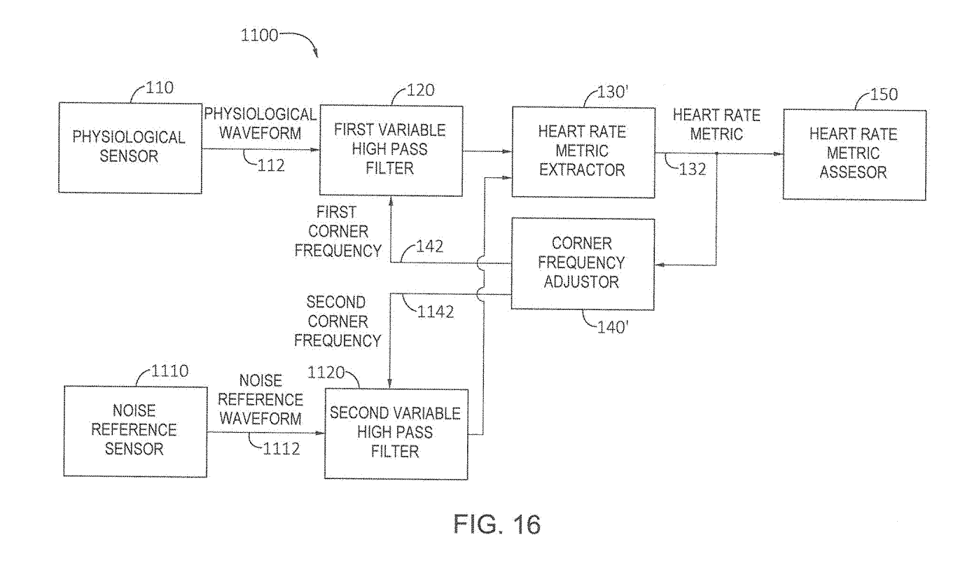

FIG. 16 is a functional block diagram of physiological signal processing systems and methods according to various embodiments described herein. Referring now to FIG. 16, these physiological signal processing systems/methods 1100 may include a physiological sensor 110 that is configured to generate a physiological waveform 112 that includes a cardiovascular signal component and a noise component therein. The noise component may be as a result of motion of the user. A variable high pass filter 120, referred to herein as a first variable high pass filter 120, is also provided.

Still referring to FIG. 16, a noise reference sensor 1110 is also provided. In some embodiments, the noise reference sensor 1110 is used to reduce and, in some embodiments to eliminate, interference from motion noise with the heart rate metric extractor accuracy. In some embodiments, the inertial sensor may comprise an accelerometer, a pressure sensor and/or a blocked channel sensor. The use of an accelerometer as a motion noise reference is described, for example, in U.S. Pat. No. 7,144,375 to Kosuda, the disclosure of which is hereby incorporated by reference herein in its entirety. A blocked channel sensor is described, for example, in U.S. Patent Application Publication No. 2014/0135596, published on May 15, 2014 to LeBoeuf et al., entitled “Form-Fitted Monitoring Apparatus for Health And Environmental Monitoring”, assigned to the Assignee of the present application; and in U.S. Patent Application Publication No. 2014/0249381, published on Sep. 4, 2014 to LeBoeuf et al., entitled “Light-Guiding Devices And Monitoring Devices Incorporating Same”, assigned to the Assignee of the present application, the disclosures of both of which are hereby incorporated by reference herein in their entirety. In brief, a “blocked channel” refers to measuring optical scatter from a non-body interface during motion. This optical scatter signal has motion information in it, but not physiological information in it. For this reason, it can be used as a noise reference sensor to attenuate motion noise from a PPG signal. Other noise reference sensors 1110 may also be employed.

As noted above, the noise reference sensor 1110 is configured to generate a noise reference waveform 1112 including the noise component of the physiological waveform 112 therein. Stated differently, in some embodiments, the noise reference sensor 1110 contains a facsimile of the motion noise that is also present in the physiological waveform 112 that is generated by the physiological sensor 110. In other embodiments, the noise reference waveform 1112 is substantially devoid of a cardiovascular signal component. As used herein, the term “substantially devoid of a cardiovascular signal component” means that there may be some remnant of the cardiovascular signal component in the noise reference waveform 1112 that is generated by the noise reference sensor 1110, but this remnant does not impact the operation of the heart rate metric extractor 130′.

Still referring to FIG. 16, a second variable high pass filter 1120 is provided, that is responsive to the noise reference waveform 1112 and is configured to high pass filter the noise reference waveform 1112 in response to a second corner frequency 1142 that is applied thereto. A heart rate metric extractor 130′ is also provided. The heart rate metric extractor 130′ may be embodied as was described above in connection with the heart rate metric extractor 130. In other embodiments, since the heart rate metric extractor 130′ is responsive to the output of the first variable high pass filter 120 and to the output of the second variable high pass filter 1120, the heart rate metric extractor may operate by obtaining a difference between the physiological waveform 112 that is high pass filtered by the first variable high pass filter 120 and the noise reference waveform 1112 that is high-pass filtered by the second variable high pass filter 1120. In some embodiments the heart rate metric extractor 130′ comprises a spectral subtractor that is configured to obtain a difference between a frequency domain representation of the physiological waveform 112 that is high-pass filtered by the first variable high pass filter 120 and a frequency domain representation of the noise reference waveform 1112 that is high-pass filtered by the second variable high pass filter 1120. In other embodiments, rather than a difference of frequency domain representations, a difference between time domain representations may be obtained by the heart rate metric extractor 130′.

Embodiments of a heart rate metric extractor 130′ that may be used herein to extract a heart rate metric in the presence of noise using a noise reference sensor are described, for example, in U.S. Patent Application Publication 2015/001898 to the present inventor Romesburg, entitled “Physiological Metric Estimation Rise And Fall Limiting”, published on Jan. 8, 2015, assigned to the Assignee of the present application; and published PCT Application WO 2013/109390 A1 to the present inventor Romesburg, entitled “Reduction Of Physiological Metric Error Due To Inertial Cadence”, published on Jul. 25, 2013, and assigned to the Assignee of the present application, the disclosures of both of which are incorporated herein by reference in their entirety as if set forth fully herein.

In other embodiments, a spectral subtraction technique as described in the above cited U.S. Pat. No. 7,144,375 may be used to extract a heart rate metric in the presence of noise using a noise reference sensor. As noted above, either time domain or frequency domain subtraction may be employed, for example, using least mean squares filters as described in a Wikipedia article entitled “Least mean squares filter”, (https://en.wikipedia.org/wiki/Least_mean_squares_filter) for least mean squares filters and normalized least mean squares filters in the time domain.

Still referring to FIG. 16, the heart rate metric extractor 130′ extracts a heart rate metric 132 using any of the above and/or other techniques. A corner frequency adjustor 140′ is responsive to the heart rate metric extractor 130′ and is configured to determine first and second corner frequencies 142, 1142 respectively, that are applied to the first and second variable high pass filters 120, 1120 respectively, based on the heart rate metric 132 that was extracted. In some embodiments the first and second corner frequencies are substantially different from one another, meaning that they differ by at least 10 percent. In other embodiments, however, the first and second corner frequencies may be a substantially same corner frequency, meaning they are within 10 percent of one another. Moreover, in some embodiments they may be identical (within the resolution of the corner frequency adjustor).

According to some embodiments of FIG. 16, since a variable high-pass filter 1120 is applied to the noise reference waveform 1112, and both variable high-pass filters 120, 1120 feed off the heart rate metric extractor 130′, the motion noise may be advantageously attenuated in the physiological waveform 112 because the noise reference waveform 1112 has the same high-pass filter applied. Thus, the noise reference waveform 1112 that is filtered by the second variable high pass filter 120 can serve as a better noise reference due to the better matching spectral characteristics to the motion noise component of the high-pass filtered physiological waveform 112.

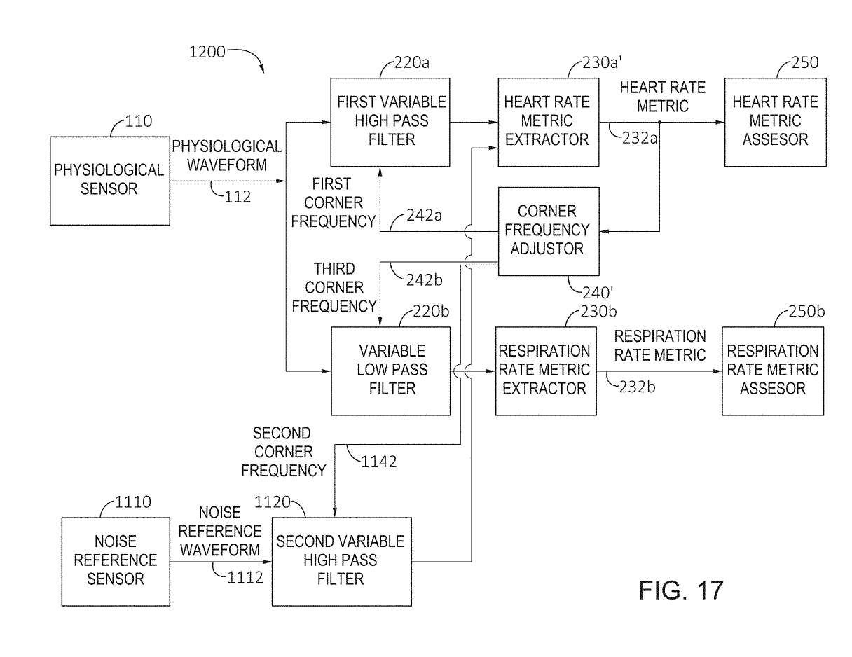

FIG. 17 is a functional block diagram of physiological signal processing systems and methods according to various other embodiments described herein. These physiological signal processing systems and methods 1200 correspond to the system/methods 200 of FIG. 2 but are supplemented by a noise reference sensor 1110 that produces a noise reference waveform 1112 that is filtered by a second variable high pass filter 1120 as was described in connection with FIG. 16. Note that in FIG. 17, the corner frequency 242b is labeled as a third corner frequency for consistency with the labeling of FIG. 16. Thus, embodiments of FIG. 17 are configured to extract cardiovascular and pulmonary physiological signal components that are included in physiological waveform 112 as provided by the physiological sensor 110. The components of FIG. 17 may operate as was described in connection with FIGS. 2 and 16, and this description will not be repeated in the interest of brevity. Moreover, when the signals are processed in the time domain, other metrics such as RRi (i.e., the interval between R waves of the QRS complex of the cardiac cycle, which may be indicated by the time-difference between consecutive R-peaks in an ECG or PPG waveform) and/or other biometrics may also be extracted.

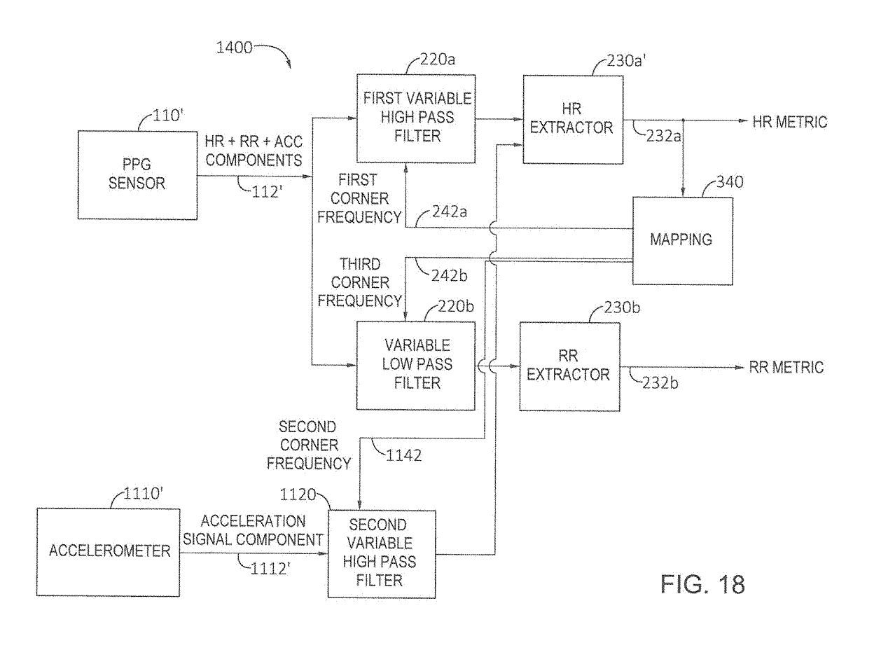

FIG. 18 is a more detailed functional block diagram of embodiments 1400 of FIG. 17 and may be used to extract a heart rate metric 232a and a respiration rate metric 232b from a PPG sensor 110′ that provides a PPG sensor waveform 112′ that includes both heart rate and respiration rate components as well as an acceleration component. An accelerometer 1110′ provides an acceleration waveform 1112′ that includes the acceleration component, to a second variable high pass filter 1120. The elements of FIG. 18 may be embodied as was described above. Accordingly, embodiments of FIG. 18 provide a physiological signal processing system that comprises a PPG sensor 110′ that is configured to generate a PPG waveform 112′ that includes a cardiovascular signal component (HR), a respiration rate signal component (RR) and an acceleration signal component (ACC). An accelerometer 1110′ is also provided that is configured to generate a an acceleration waveform 1112′ including the acceleration signal component therein. A first variable high pass filter 220a, a second variable high pass filter 1120, a variable low-pass filter 220b, a heart rate metric extractor 230a′, a respiration rate extractor 230b and a mapping function 340 are provided as was described, for example, in connection with FIGS. 4 and 17. They will not be described again in the interest of brevity.

Additional descriptions of various embodiments described herein will now be provided. Specifically, embodiments of FIGS. 16-18 add a noise reference sensor 1110 which may be an accelerometer 1110′. For purposes of the present embodiments, the primary purpose of the noise reference sensor 1110/1110′ is to provide a noise reference waveform/acceleration waveform 1112/1112′ that may be filtered and used for heart rate metric extraction. In some embodiments, however, the noise reference sensor/accelerometer 1110/1110′ may have a secondary purpose such as counting the number of steps or step rate.

Ideally, the noise reference waveform/acceleration waveform 1112/1112′ contains a facsimile of the motion noise that is in the physiological waveform 112/112′ but does not substantially contain the heart rate or respiration rate components thereof. Thus, the noise reference sensor/accelerometer 1110/1110′ produces a noise reference waveform/acceleration waveform which is also manifested in the physiological waveform 112/112′. Both the physiological waveform 112/112′ and the noise reference waveform/acceleration waveform 1112/1112′ are filtered by variable high-pass filters 120/220a/1120. In some embodiments substantially the same or an identical corner frequency is used, so as to match the motion component in both waveforms 112/112′ and 1112/1112′. Viewed differently, various embodiments described herein may provide a feedback loop from the heart rate metric extractor 130′/230a′ to the first and second variable high-pass filters 120/220a/1120, via the corner frequency adjustor 140′/240′/340.

Various embodiments have been described herein with reference to block diagrams and a flowchart of methods, apparatus (systems and/or devices) and/or computer program products. It is understood that a block of the block diagrams and/or flowchart, and combinations of blocks in the block diagrams and/or flowchart, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart, and thereby create means (functionality), structure and/or methods for implementing the functions/acts specified in the block diagrams and/or flowchart.

These computer program instructions may also be stored in a computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks.

A tangible, non-transitory computer-readable medium may include an electronic, magnetic, optical, electromagnetic, or semiconductor data storage system, apparatus, or device. More specific examples of the computer-readable medium would include the following: a portable computer diskette, a random access memory (RAM) circuit, a read-only memory (ROM) circuit, an erasable programmable read-only memory (EPROM or Flash memory) circuit, a portable compact disc read-only memory (CD-ROM), and a portable digital video disc read-only memory (DVD/Blu-Ray™).

The computer program instructions may also be loaded onto a computer and/or other programmable data processing apparatus to cause a series of operational steps to be performed on the computer and/or other programmable apparatus to produce a computer-implemented process or method such that the instructions which execute on the computer or other programmable apparatus provide steps for implementing the functions/acts specified in the block diagrams and/or flowchart.

Accordingly, the invention may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the blocks. For example, two blocks shown in succession may in fact be executed substantially concurrently or the blocks may sometimes be executed in the reverse order, depending upon the functionality/acts involved. Moreover, the functionality of a given block of the block diagrams and/or flowchart may be separated into multiple blocks and/or the functionality of two or more blocks of the block diagrams and/or flowchart may be at least partially integrated. Finally, other blocks may be added/inserted between the blocks that are illustrated.

Many different embodiments have been disclosed herein, in connection with the above description and the drawings. It will be understood that it would be unduly repetitious and obfuscating to literally describe and illustrate every combination and subcombination of these embodiments. Accordingly, the present specification, including the drawings, shall be construed to constitute a complete written description of all combinations and subcombinations of the embodiments described herein, and of the manner and process of making and using them, and shall support claims to any such combination or subcombination.

In the drawings and specification, there have been disclosed embodiments of the invention and, although specific terms are employed, they are used in a generic and descriptive sense only and not for purposes of limitation, the scope of the invention being set forth in the following claims.

What is claimed is:

1. A physiological signal processing system comprising:
a physiological sensor that is configured to generate a physiological waveform that includes a cardiovascular signal component and a noise component therein;
a noise reference sensor that is configured to generate a noise reference waveform including the noise component therein;
a first variable high pass filter that is responsive to the physiological waveform and that is configured to high pass filter the physiological waveform in response to a first corner frequency that is applied thereto;
a second variable high pass filter that is responsive to the noise reference waveform and that is configured to high pass filter the noise reference waveform in response to a second corner frequency that is applied thereto;
a heart rate metric extractor that is responsive to the first and second variable high pass filters and that is configured to extract a heart rate metric from the physiological waveform that is high pass filtered by the first variable high pass filter and from the noise reference waveform that is high pass filtered by the second variable high pass filter; and
a corner frequency adjustor that is responsive to the heart rate metric extractor and that is configured to determine the first and second corner frequencies that are applied to the first and second variable high pass filters, respectively, based on the heart rate metric that was extracted.

2. A physiological signal processing system according to claim 1 wherein the noise component comprises a motion component and the reference sensor comprises an inertial sensor.

3. A physiological signal processing system according to claim 2 wherein the inertial sensor comprises an accelerometer, a pressure sensor and/or a blocked channel sensor.

4. A physiological signal processing system according to claim 1 wherein the noise reference waveform is substantially devoid of the cardiovascular signal component.

5. A physiological signal processing system according to claim 1 wherein the heart rate metric extractor is configured to obtain a difference between the physiological waveform that is high pass filtered by the first variable high pass filter and the noise reference waveform that is high pass filtered by the second variable high pass filter.

6. A physiological signal processing system according to claim 5 wherein the heart rate metric extractor comprises a spectral subtractor that is configured to obtain a difference between a frequency domain representation of the physiological waveform that is high pass filtered by the first variable high pass filter and a frequency domain representation of the noise reference waveform that is high pass filtered by the second variable high pass filter.

7. A physiological signal processing system according to claim 1 wherein the first and second corner frequencies comprise a substantially same corner frequency.

8. A physiological signal processing system according to claim 1, wherein the physiological waveform also includes a pulmonary signal component therein, the physiological signal processing system further comprising:
a variable low pass filter that is responsive to the physiological waveform and that is configured to low pass filter the physiological waveform in response to a third corner frequency that is applied thereto; and
a respiration rate metric extractor that is responsive to the variable low pass filter and that is configured to extract a respiration rate metric from the physiological waveform that is filtered by the variable low pass filter;
wherein the corner frequency adjustor is further configured to determine the third corner frequency that is applied to the variable low pass filter based on the heart rate metric that was extracted.

9. A physiological signal processing system according to claim 1 wherein the physiological waveform comprises an electrical physiological waveform including an electroencephalogram (EEG), an electrocardiogram (ECG) and/or a radio frequency (RF) waveform, an electro-optical physiological waveform including a photoplethysmograph (PPG) waveform, an electro-photoacoustic waveform including a photoacoustic waveform, an electro-mechanical physiological waveform including an auscultation waveform, a piezo sensor waveform and/or an accelerometer waveform, and/or an electro-nuclear physiological waveform.

10. A physiological signal processing system according to claim 1 wherein the first and second variable high pass filters each comprises a single high pass filter having an adjustable corner frequency.

11. A physiological signal processing system according to claim 1 wherein the first and second variable high pass filters each comprises a plurality of high pass filters, a respective one of which includes a different value of the corner frequency, and wherein the corner frequency adjustor is configured to select one of the plurality of high pass filters that corresponds to the corner frequency that is determined.

12. A physiological signal processing system according to claim 1 wherein the first and second variable high pass filters each comprises a variable digital high pass filter having a plurality of delay taps and wherein the corner frequency corresponds to a number of the plurality of delay taps that are selected to filter the physiological waveform.

13. A physiological signal processing system according to claim 12 wherein the corner frequency adjustor comprises a mapping system that is configured to map the heart rate metric that is extracted into the number of the delay taps that are selected to high pass filter the physiological waveform.

14. A physiological signal processing system according to claim 1 wherein the corner frequency adjuster is configured to reduce locking on an erroneous heart rate metric.

15. A physiological signal processing system according to claim 1 wherein the corner frequency adjuster is configured to initially set predetermined first and second corner frequencies corresponding to a predetermined heart rate prior to determining the first and second corner frequencies that are applied to the first and second variable high pass filters, respectively.

16. A physiological signal processing system according to claim 15 wherein the predetermined heart rate is a resting heart rate.

17. A physiological signal processing system according to claim 15 wherein the corner frequency adjuster is configured to initially set the predetermined first and second corner frequencies corresponding to the predetermined heart rate until the heart rate metric extractor locks on a heart rate in the physiological waveform.

18. A physiological signal processing system according to claim 15 wherein the corner frequency adjuster is configured to reset the predetermined first and second corner frequencies corresponding to the predetermined heart rate in response to determining that the physiological sensor is no longer responsive to a source of the physiological waveform.

19. A physiological signal processing system according to claim 1 wherein the corner frequency adjuster is configured to set predetermined first and second corner frequencies corresponding to a predetermined heart rate in response to determining that the physiological sensor is not responsive to a source of the physiological waveform.

20. A physiological signal processing system according to claim 1 wherein the corner frequency adjuster is configured to determine the first and second corner frequencies that are applied to the first and second variable high pass filters, respectively, by applying a margin to the heart rate metric.

21. A physiological signal processing system according to claim 1 further comprising:
a physiological metric assessor that is responsive to the heart rate metric extractor and that is configured to process the heart rate metric to generate at least one physiological assessment.

22. A physiological signal processing system comprising:
a photoplethysmograph (PPG) sensor that is configured to generate a PPG waveform that includes a cardiovascular signal component and an acceleration signal component therein;
an accelerometer that is configured to generate an accelerometer waveform including the acceleration component therein;
a first variable high pass filter that is responsive to the PPG waveform and that is configured to high pass filter the PPG waveform in response to a first corner frequency that is applied thereto;
a second variable high pass filter that is responsive to the accelerometer waveform and that is configured to high pass filter the accelerometer waveform in response to a second corner frequency that is applied thereto;
a heart rate metric extractor that is responsive to the first and second variable high pass filters and that is configured to extract a heart rate metric from the PPG waveform that is high pass filtered by the first variable high pass filter and from the accelerometer waveform that is high pass filtered by the second variable high pass filter; and
a corner frequency adjustor that is responsive to the heart rate metric extractor and that is configured to determine the first and second corner frequencies that are applied to the first and second variable high pass filters, respectively, based on the heart rate metric that was extracted.

23. A physiological signal processing method for a physiological waveform that includes a cardiovascular signal component and a noise component therein, and a noise reference waveform including the noise component therein, the physiological signal processing method comprising:
high pass filtering the physiological waveform in response to a first adjustable high pass filter corner frequency;
high pass filtering the noise reference waveform in response to a second adjustable high pass filter corner frequency;
extracting a heart rate metric from the physiological waveform that is high pass filtered and from the noise reference waveform that is high pass filtered; and
determining the first and second adjustable high pass filter corner frequencies from the heart rate metric that was extracted.
